(19) United States
(12) Patent Application Publication
(10) Pub. No.: US 2014/0364841 A1
(43) Pub. Date: Dec. 11, 2014

(54) CRYOLIPOLYIS DEVICE HAVING A CURVED APPLICATOR SURFACE

(71) Applicant: Andrew Kornstein, Fairfield, CT (US)

(72) Inventor: Andrew Kornstein, Fairfield, CT (US)

(21) Appl. No.: 13/261,891

(22) PCT Filed: Nov. 14, 2012
(86) PCT No.: PCT/US12/65067

§ 371 Date: May 14, 2014

Related U.S. Application Data
(60) Provisional application No. 61/559,605, filed on Nov. 14, 2011.

(51) Int. Cl. A61B 18/02

(52) U.S. Cl. CPC A61B 18/02; A61B 2018/00291
USPC 606/20

(57) ABSTRACT

An applicator for treating lipid-rich cells disposed under a cutaneous layer includes a vacuum cup defining an interior cavity. The vacuum cup has a first concave contour that defines a mouth of the interior cavity. At least a first cutout extends through a first sidewall of the vacuum cup. At least a first cooling unit is disposed in the first cutout. The cooling unit has a second concave contour. The cooling unit is configured for heat transfer with respect to the lipid-rich cells when the first and second contours engage the cutaneous layer.

### BACKGROUND

Excess body fat, or adipose tissue, can detract from personal appearance and athletic performance, and can pose significant health risks by increasing the likelihood of developing various types of diseases, for example, heart disease, high blood pressure, osteoarthritis, cancer, bronchitis, hypertension, diabetes, deep-vein thrombosis, pulmonary emboli, varicose veins, gallstones, and hernias.

Surgical procedures such as liposuction have been employed to remove excess body fat. Due to its invasive nature, recovery time, potential complications and the cost of such surgical procedures, the demand for safe and effective non-invasive alternatives for body contouring have grown with the public's demand. Many non-invasive body contouring procedures exist in an attempt to remove or reduce adipose cells. These include topical agents, massages, acupuncture, weight-loss drugs, exercise, dieting, and applying heat to subcutaneous lipid-rich areas. However, each of the methods have limitations making the methods ineffective or impractical in certain circumstances.

Studies have shown that cooling subcutaneous lipid-rich areas results in crystallization of cytoplasmic lipid deposits within adipose cells resulting in cell damage or cell death. Immune cells engulf the affected adipose cells and eliminate them from the body. The remaining fat layer condenses, reducing fat volume at the target area. The apparatus that is used to remove heat from the subcutaneous lipid-rich cells is often referred to as a cryolipolyis device.

Cryolipolyis devices may employ different types of applicators that are placed against the patient's epidermis to cool various target areas of the patient. One type of applicator is a vacuum applicator, which includes a vacuum cup that has a pair of cutouts in which thermal conductors are positioned. A heat removal source is coupled to the exterior surface of the thermal conductors. In operation, the vacuum applicator is placed against the cutaneous layer of the patient and the suction source is activated to draw the cutaneous layer into the interior cavity of the vacuum cup. The removal source is then activated to remove heat from the lipid-rich cells.

### SUMMARY

In accordance with one aspect of the invention, an applicator for treating lipid-rich cells disposed under a cutaneous layer is provided. The applicator includes a vacuum cup defining an interior cavity. The vacuum cup has a first concave contour that defines a mouth of the interior cavity. At least a first cutout extends through a first sidewall of the vacuum cup. At least a first cooling unit is disposed in the first cutout. The cooling unit has a second concave contour. The cooling unit is configured for heat transfer with respect to the lipid-rich cells when the first and second contours engage the cutaneous layer.

In accordance with another aspect of the invention, a treatment device for treating lipid-rich cells disposed under a cutaneous layer is provided. The treatment device includes a flexible member, at least one cooling unit and a support member. The flexible member has an inner and outer surface. The inner surface defines an interior cavity. The flexible member includes a first distal surface coupling the inner surface to the outer surface. The first distal surface is configured to engage with the cutaneous layer. The first distal surface has a concave curvature. The cooling unit is coupled to the flexible member. The cooling unit has a thermally conductive member configured to contact the cutaneous layer when the cutaneous layer is drawn into the interior cavity when a vacuum is established therein. The cooling unit has an outer housing with a second distal surface that is also configured to engage with the cutaneous layer when the first distal surface of the flexible member engages with the cutaneous layer. The second distal surface of the outer housing has a concave curvature. The support member is coupled to a proximal end of the flexible member.

In accordance with yet another aspect of the invention, an applicator is provided for treating lipid-rich cells disposed under a cutaneous layer. The applicator includes a vacuum cup having first and second opposing sidewalls defining an interior cavity. The vacuum cup includes a first distal surface that defines a mouth of the interior cavity. At least a first cutout extends through the first sidewall of the vacuum cup; A cooling unit is disposed in the first cutout. The cooling unit is configured for heat transfer with respect to the lipid-rich cells when the first distal surface engages the cutaneous layer. at least one expansion joint is disposed in at least one of the sidewalls of the vacuum cup for adjusting at least one dimension of the interior cavity.

### BRIEF DESCRIPTION OF THE DRAWINGS

FIG. 1 is a simplified, schematic diagram of a cryolipolyis device having a treatment device with a curved applicator.

FIG. 2 is a front perspective view of the curved applicator shown in FIG. 1.

FIG. 3 is a perspective view illustrating various aspects of the treatment device shown in FIG. 1

FIG. 4 is a perspective view of the vacuum cup employed in the curved applicator of FIG. 2.

FIG. 5 shows the treatment device of FIG. 1 when applied to a patient's hip.

FIG. 6 is a schematic cross-sectional view of a cooling unit that may be employed by the treatment device of FIG. 1.

FIG. 7 is a side view of an alternative embodiment of the treatment device.

FIG. 8 is a perspective view of another alternative embodiment of the treatment device.

### DETAILED DESCRIPTION

The cryolipolyis device described herein is suitable for treating a subject's subcutaneous adipose tissue, such as by cooling. The “subcutaneous tissue” can include tissue lying beneath the dermis and includes subcutaneous fat, or adipose tissue that may be composed primarily of lipid-rich cells, or adipocytes. When cooling subcutaneous tissues to a temperature lower than about 37 C., subcutaneous lipid-rich cells can be affected selectively. In general, the epidermis and dermis of the subject lack lipid-rich cells compared to the underlying lipid-rich cells forming the adipose tissue. Because non-lipid-rich cells usually can withstand colder temperatures better than lipid-rich cells, the subcutaneous lipid-rich cells can be affected selectively without affecting the non-lipid-rich cells in the dermis, epidermis and other surrounding tissue. In some embodiments, the cryolipolyis device can apply cooling temperatures to the epidermis of the subject in a range of from about −20 C. to about 20 C.

The cryolipolyis device can damage, injure, disrupt or otherwise reduce subcutaneous lipid-rich cells generally without collateral damage to non-lipid-rich cells in the treatment target area. In general, it is believed that lipid-rich cells can be affected selectively (e.g., damaged, injured, or disrupted) by exposing such cells to low temperatures that do not so affect non-lipid-rich cells to the same extent or in the same manner. As a result, lipid-rich cells, such as subcutaneous adipose tissue, can be damaged while other cells in the same region are generally not damaged even though the non-lipid-rich cells at the surface are subject to even lower temperatures. The mechanical energy provided by the applicator as well as manual pressure massage may further enhance the effect on lipid-rich cells by mechanically disrupting the affected lipid-rich cells.

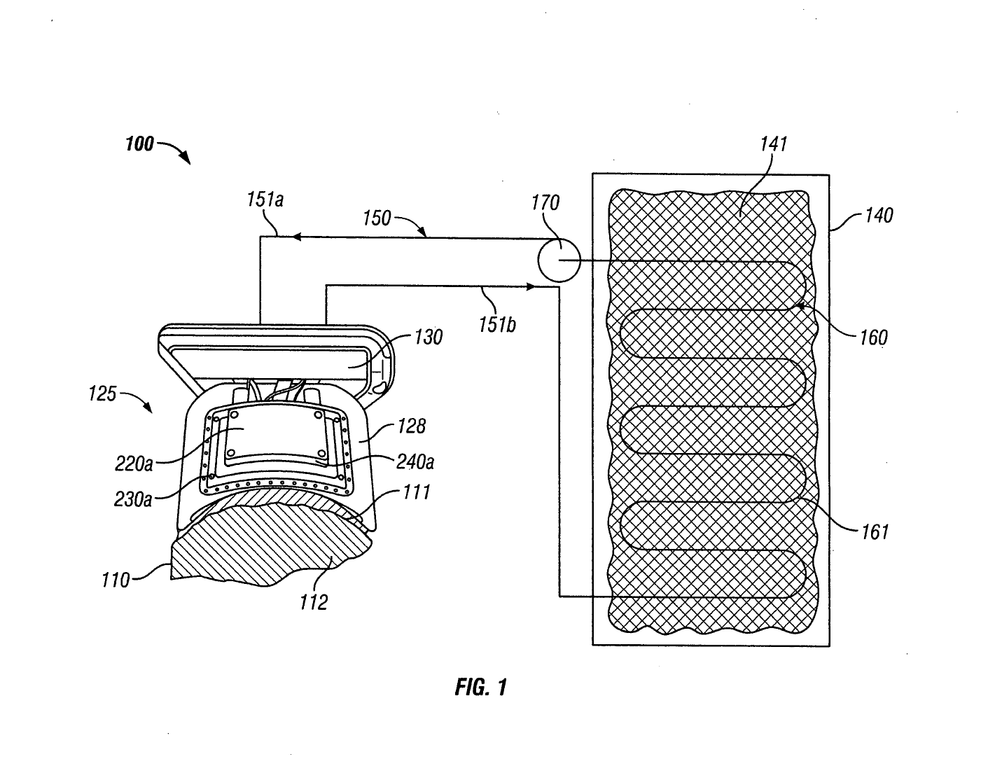

FIG. 1 is a simplified, schematic diagram of a cryolipolyis device 100 having a treatment device 125 operatively coupled to a coolant vessel 140 to cool human tissue 110. In particular, the device 100 is configured to cool subcutaneous, lipid-rich tissue 112, without damaging the overlying dermis 111. The treatment device 125 is coupled to the coolant vessel 140 by a heat transfer conduit 150 that carries a heat transfer fluid. Accordingly, the heat transfer conduit 150 includes a supply portion 151a that directs the heat transfer fluid to the treatment device 125, and a return portion 151b that receives heat transfer fluid exiting the treatment device 125. The heat transfer fluid is propelled through the heat transfer conduit 150 by a fluid driver 170, e.g., a pump or other suitable device. The heat transfer conduit 150 is typically insulated to prevent the ambient environment from heating the heat transfer fluid. Other elements of the device (aside from the cooling surface of the applicator of the treatment device 125 in contact with the tissue 110) are also insulated from the ambient environment to prevent heat loss and frost formation. Examples of suitable heat transfer fluid include, without limitation, water, glycol, synthetic heat transfer fluid, oil and a refrigerant.

The heat transfer conduit 150 is connected to a heat exchanger 160 having a heat exchanger conduit (e.g., tubing) 161 that is positioned within or at least partially within the coolant vessel 140. The coolant vessel 140 contains a coolant 141 that is in close thermal contact with the heat exchanger 160, but is isolated from direct fluid contact with the heat transfer fluid contained within the heat exchanger tubing 161. Accordingly, the heat exchanger 160 facilitates heat transfer between the heat transfer fluid and the coolant 141, while preventing these fluids from mixing. As a result, the coolant 141 can be selected to have a composition different than that of the heat transfer fluid.

In some embodiments, instead of using coolant 141, other cooling devices capable of removing heat may be employed, such as a refrigeration unit, a cooling tower, a thermoelectric chiller or cooler. Regardless of the technology that is employed, the cooling device may be incorporated into, or otherwise operatively associated with, a treatment unit that includes additional components such as a processor, an input device, an output device, a control panel and power supply. The processor may monitor process parameters via sensors placed proximate to the treatment device 125 through a signal line to, among other things, adjust the heat removal rate based on process parameters. The processor may further monitor process parameters to adjust the treatment device 125 based on the process parameters. The input device may be, for example, a keyboard, a mouse, a touch screen, a push button, a switch, a potentiometer, any combination thereof, and any other device or devices suitable for accepting user input. The output device may include, for example, a display or touch screen, a printer, a medium reader, an audio device, a visual device, any combination thereof, and any other device or devices suitable for providing user feedback.

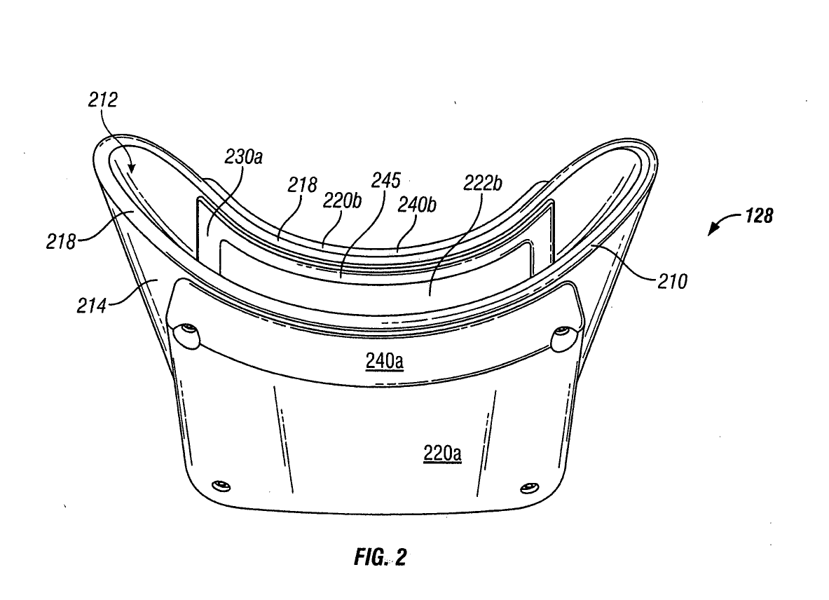

In FIG. 1, the treatment device 125 is shown to include an applicator 128 and an applicator support 130. The applicator 128 is coupled to the applicator support 130 at its proximal end. Details concerning aspects of the treatment device are shown in FIGS. 2-4. In FIGS. 1-4 and the figures that follow, like elements are denoted by like reference numerals.

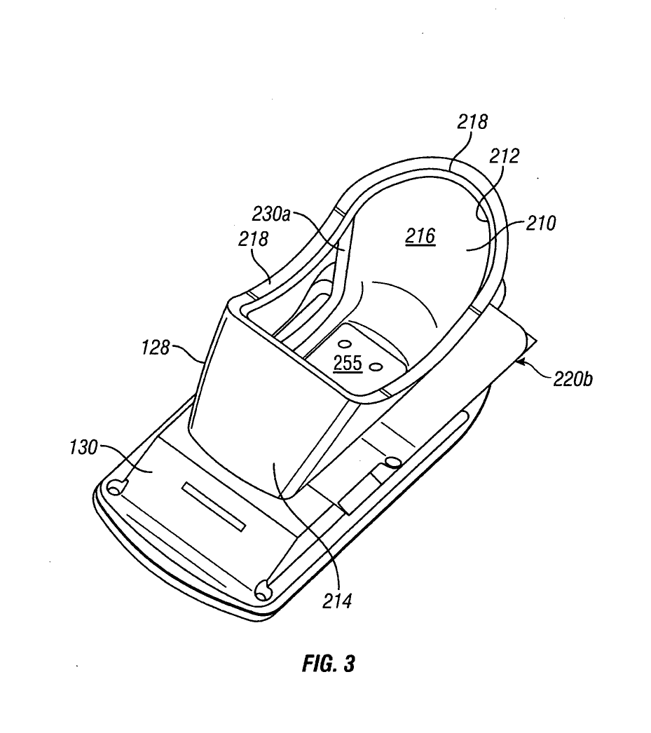

FIG. 2 shows the applicator 128 itself, which includes a flexible vacuum cup 210 and cooling units 220a and 220b. The vacuum cup 210 includes an interior surface 212 and an exterior surface 214. The interior surface 212 defines an interior cavity 216 (see FIG. 3) in which a vacuum may be drawn. The flexible vacuum cup 210 has a distal end defining the mouth of the interior cavity 216, which has a concave contoured distal surface 218 joining the interior and exterior surfaces 212 and 214. The distal surface 218 contacts the epidermis of the patient when the treatment device 125 is applied thereto.

The vacuum that is applied by the treatment device 125 may be used to assist in forming a contact between the treatment device and the patient's epidermis. The vacuum may also be used to impart mechanical energy during treatment. Imparting mechanical vibratory energy to a target area by, e.g., repeatedly applying and releasing a vacuum to the subject's tissue, or for instance, modulating a vacuum level applied to the subject's tissue to create a massage action during treatment.

In some embodiments, some or all of the functionality of the control panel referred to above may be located on the applicator support 130 so as to be readily accessible to the operator of the cryolipolyis device. The control panel may provide the operator with the ability to control and/or monitor treatment. For example, a first ON/OFF button may toggle the initiation or termination of a treatment and a second ON/OFF button may actuate a pump (not shown) for drawing a vacuum in the interior cavity 216. Indicator lights may provide a visual indication of, for example, whether a treatment is proceeding and/or whether the vacuum pump is activated.

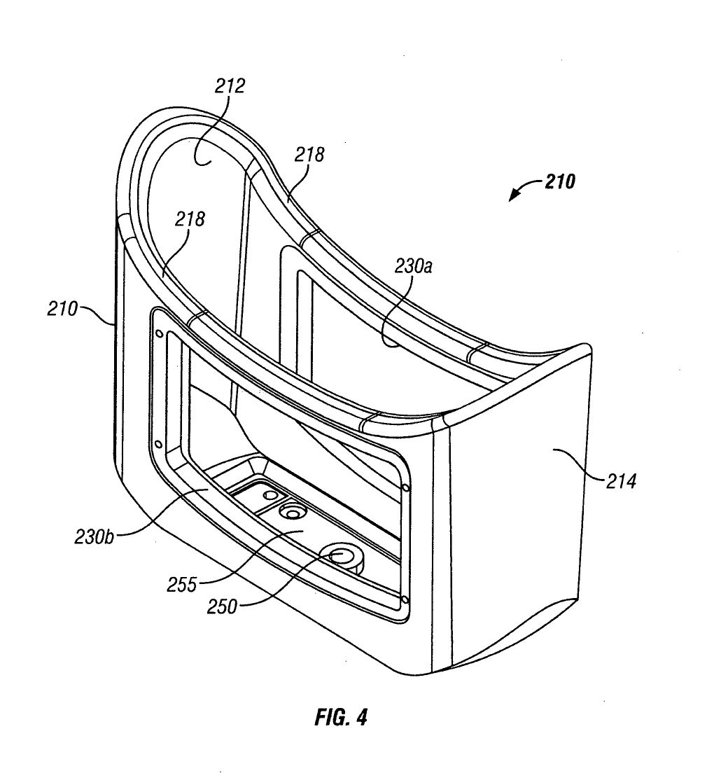

As seen in FIGS. 3 and 4, the applicator 128 and applicator support 130 may be operatively coupled to one another by a mounting plate 255 located at the proximal end of the interior cavity 216. The mounting plate 255 may be integrally formed with the vacuum cup 210 or separately coupled to the vacuum cup 210. An aperture 250 (see FIG. 4) in the mounting plate 255 provides a passage for drawing a vacuum in the interior cavity 216. One or more fasteners may releasably secure the mounting plate 255 to the housing applicator support 130. In other embodiments, adhesive or another type of fastener may be used to couple the applicator 128 to the applicator support 130 either with or without using the mounting plate 225. Additional apertures (not shown) may be located in the mounting plate 255 to allow heat transfer conduit 150 and sensor wires to pass through the applicator support 130 and be coupled to the cooling units 220a and 220b.

The cooling units 220a and 220b are located in opposing sidewalls of the flexible vacuum cup 210. As shown in FIG. 4, the vacuum cup 210 may include cutouts located in opposing sidewalls each being defined by a support frame 230a and 230b. The cooling units 220a and 220b are configured for heat transfer with respect to the lipid-rich cells when the contoured surface of the vacuum cup 210 contacts the cutaneous layer, which is drawn into the interior cavity 216 upon application of a vacuum within the vacuum cup 210. More particularly, the cooling units 220a and 22b each have a thermal conductor exposed to the interior cavity 216. One of the thermal conductors, thermal conductor 222b, is visible in FIG. 2. While cooling units 220a and 220b may employ any suitable technology in order to facilitate heat transfer, one example of a cooling unit 220a and 220b will be illustrated below which employs thermoelectric elements and a fluidic cryoprotectant.

In some embodiments the support frames 230a and 230b include rigid metal polygons, e.g., rectangles or squares with an intervening hinge of flexible material around which the flexible vacuum cup 210 may be molded. Accordingly, the support frames 230a and 230b may include a number of apertures, grooves, or other recesses into which the material of the flexible vacuum cup 210 may flow during a molding process to provide a strong connection between the support frames 230a and 230b and the vacuum cup 210. Alternatively, the support frames 230a and 230b can be adhered, welded or otherwise coupled to the flexible vacuum cup 210 in the cutouts. The cooling units 220a and 220b can each be secured to its respective support frame 230a and 230b by any suitable means, such as fasteners (e.g., screws), adhesive, welding or the like.

In some embodiments the cooling units 220a and 220b have an outer housing with distal surfaces 240a and 240b (see FIG. 2), respectively, which also have a concave contour. Like distal surface 218, distal surfaces 240a and 240b also contact the epidermis of the patient when the treatment device is applied thereto. That is, surfaces 218, 240a and 240b, which are all concave in shape, all face in a common direction so that they can contact the epidermis when applied to the patient.

As shown in FIGS. 2 and 4, the concavity of the distal surfaces 240a and 240b may be the same as the concavity of the distal surface 218. Likewise, in order to establish a secure, fluid-tight connection, the segments of the support frames 230a and 230b which are respectively secured to the surfaces 240a and 240b have the same concave curvature as the surfaces 240a and 240b. As also shown, the surfaces 240a and 240b may be offset in the proximal direction from the surface 218 by a distance, for example, of about one-half to three-quarters of an inch. It should be noted that while the cooling units are shown to have a concave curvature on the distal end of their housings, in some embodiments the internal components of the cooling units may have the same curvature. Most notably, the thermal conductor 222b that contacts the patient's epidermis when drawn into the cavity by the vacuum cup may have a concave curvature.

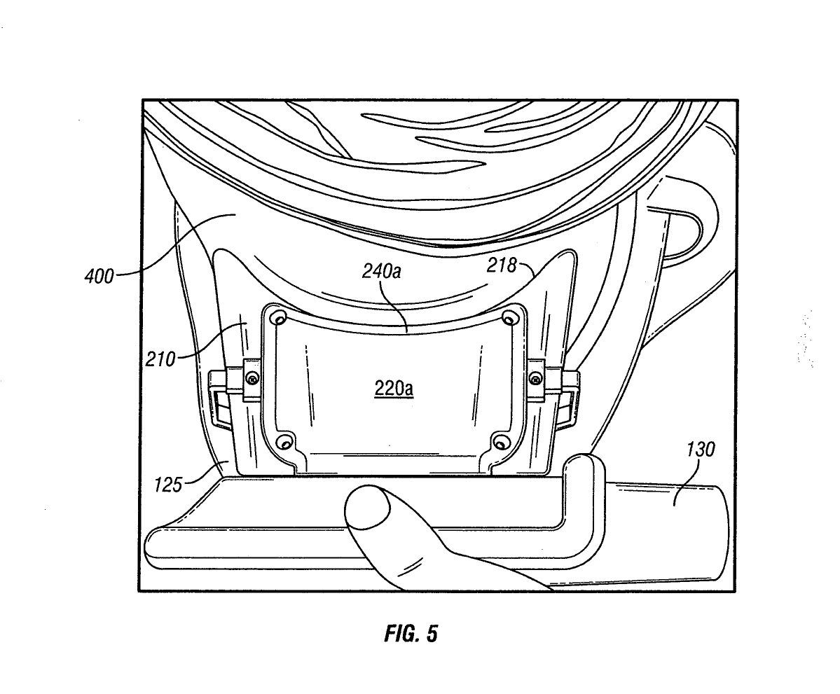

By employing cooling units 220a and 220b having curved distal surfaces as described above, the applicator 128 can better contact the epidermis of the patient, particularly those curved regions of the patient's body where epidermis elasticity is relatively poor, such as the inner thigh, the anterior and posterior axillary folds, the lateral hips, inner knees and the suprapatellar region. FIG. 5 shows the treatment device 125 when applied to a patient's hip 400. As shown, the distal surface 218 of the vacuum cup 210 and the distal surfaces 240a and 240b of the cooling units make good contact with the curved portion of the hip 400 to which the applicator 128 is applied. In contrast, an applicator in which these surfaces of the cooling units are linear is better suited to flat, two-dimensional regions on the patient's body, such as the abdomen, flanks and bras strap rolls.

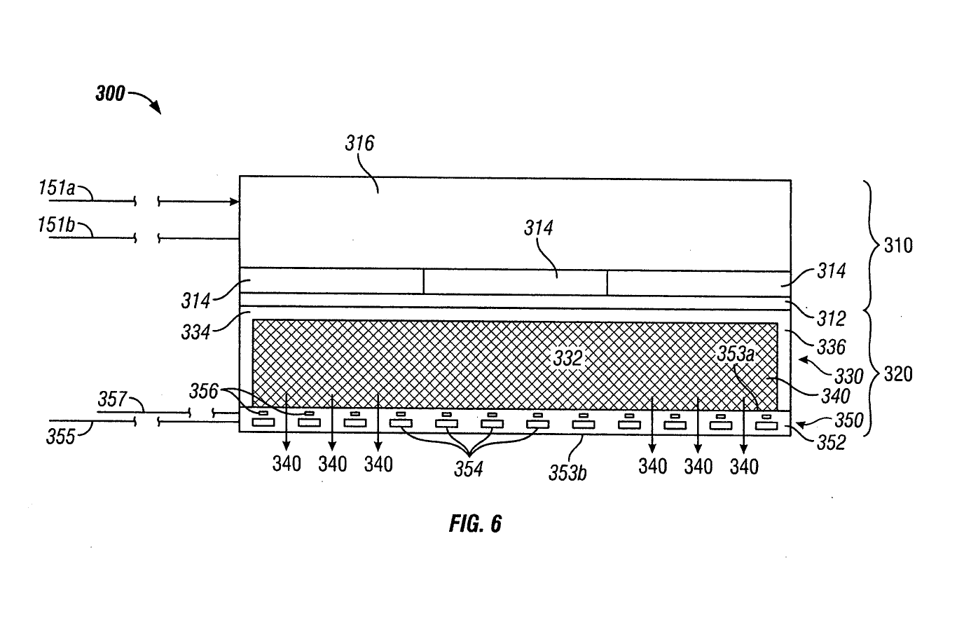

FIG. 6 is a schematic cross-sectional view of a cooling unit 300 that may be used for one or both of the cooling units 220a and 220b in applicator 128. The cooling unit includes a cooler 310 and an interface assembly 320 operably coupled to the cooler. The cooler 310 includes a plate 312 that has a high thermal conductivity, one or more Thermoelectric Elements (TEEs) 314 and a coolant chamber 316. As explained above with reference to FIG. 1, a coolant can recirculate through the coolant chamber 316 via inlet and outlet lines 151b and 151a, respectively, and the TEEs 314 can selectively heat and/or cool relative to the temperature of the coolant in the coolant chamber 316 to control the temperature over relatively large areas of the cooling plate 312. Other embodiments of the cooling unit 310 do not include the TEEs 314 such that the coolant chamber 316 extends to the cold plate 312. In either case the cooling unit 310 provides a heat sink that cools the interface assembly 320.

The interface assembly 320 further controls the heat flux through a plurality of smaller zones and delivers a cryoprotectant to the target area. In one embodiment, the interface assembly 320 includes a cryoprotectant container 330 having a cavity 332 that contains a cryoprotectant 340 and an interface element 350 through which the cryoprotectant 340 can flow. The cryoprotectant container 330 can be a rigid or flexible vessel having a back panel 334 facing the cooling unit 310 and a sidewall 336 projecting from the back panel 334. The interface element 350 can be attached to the sidewall 336 to enclose the cavity 332. The interface element 350 can include a contact member 352 having a backside 353a in contact with the cryoprotectant 340 and a front side 353b configured to contact the epidermis of the patient. The contact member 352 can be a flexible barrier (e.g., membrane) such as a porous sheet of a polymeric material or a foil with small holes, a mesh, fabric or other suitable material through which the cryoprotectant 340 can flow from the backside 353a to the front side 353b. In other embodiments, the contact member 352 can be a substantially rigid barrier that is thermally conductive and configured to allow the cryoprotectant 340 to pass from the front side 353a to the backside 353b. A rigid, thermally conductive contact member, for example, can be a plate with holes or a panel made from a porous metal material. Suitable materials for a rigid contact member 352 include aluminum, titanium, stainless steel, or other thermally conductive materials.

In some embodiments, the interface element 350 further includes an array of heating elements 354 carried by the contact member 352. The individual heating elements 354 can be arranged in a grid or other type of pattern, and each heating element 354 is independently controlled relative to the other heating elements to provide control of the heat flux through smaller, discrete zones at the interface between the target area and the interface element 350. The heating elements 354, for example, can be micro-heaters electrically coupled to a power source via a cable 355 such that the controller can selectably address individual heating elements 354. The interface element 350 can further include a plurality of temperature sensors 356 carried by the contact member 352. The temperature sensors 356 may be arranged in an array such that one or more temperature sensors can measure the heat flux through the heat flux zones associated with one or more individual heating elements 354. The temperature sensors 356 can be electrically coupled to a control unit via a cable (not shown) in a manner similar to the heating elements 354.

The various elements of the cooling units 220a and 220b are configured to resist deformation such as bowing while a vacuum is drawn into the interior cavity 216 of the vacuum cup 210 so that the front side 353b of the interface element 350 can remain in thermal contact with the epidermis of the patient. Moreover, as previously mentioned, some or all of these elements of the cooling units may have an edge with a concave curvature that matches the concave curvature of the contact surfaces of the cooling unit housings in which they are located. In particular, the contact member 352, which contacts the epidermis of the patient, may have an edge with a concave curvature. This edge is indicated by reference numeral 245 in FIG. 2. While the illustrative applicator shown herein includes two cooling units, more generally the interior cavity 216 of the vacuum cup 210 may be provided with a single cooling surface or a plurality of cooling surfaces disposed at discrete locations anywhere around the interior cavity, or the interior cavity may be partially or entirely provided with cooling surface(s).

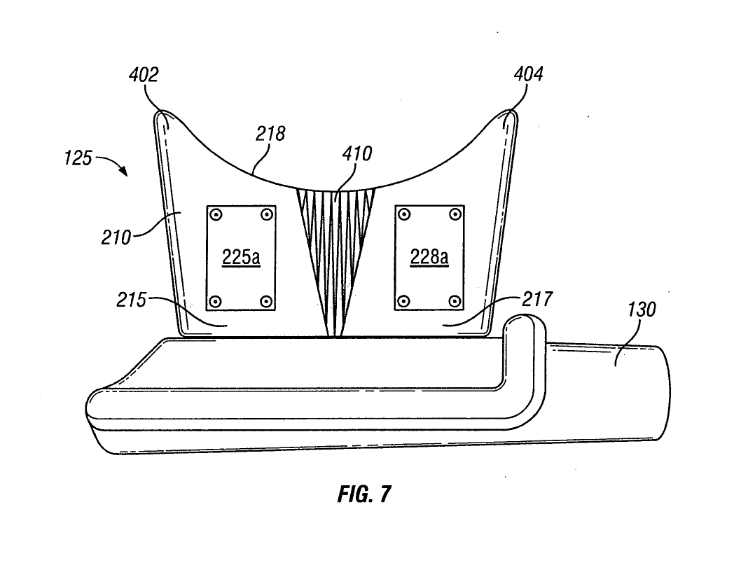

In some circumstances that arise clinically it may be advantageous to adjust the dimensions of the interior cavity 216, which would directly influence the size and shape of the contoured distal surface 218, the length of the vacuum cup 210 between its most remote ends (remote ends 402 and 404 in FIG. 7) and the distance or gap between cooling units 220a and 220b on opposing sides of the vacuum cup. By modulating the length of the vacuum cup the applicator can accommodate larger circumferential surfaces where adipose tissue resides. Likewise, by modulating the gap between cooling units the applicator can accommodate wider rolls of adipose tissue, therefore making the technology available to more potential patients. For this purpose in some embodiments the vacuum cup 210 may be provided with one or more expansion joints to better accommodate different arcs of curved surfaces as well as larger or smaller cutaneous and adipose body rolls. One example of a treatment device having such an expandable applicator is shown in FIG. 7.

The treatment device shown in FIGS. 1-6 has two cooling units, each disposed on opposing sides of the vacuum cup, which each have a thermal conductor exposed to the interior cavity 216 that contacts the patient's skin. However, the treatment device 125 having an expandable applicator shown in FIG. 7 includes two separate cooling units disposed on each side of the vacuum cup 210. In the side view of FIG. 7 only two of the cooling units, cooling units 225a and 228a are visible. The opposing side of the vacuum cup 210 may be similarly provisioned with two cooling units.

With continued reference to FIG. 7, an expansion joint 410 may be situated between the cooling units 225a and 228a. The expansion joint 410 allows the dimensions of the vacuum cup's mouth to be adjusted by the practitioner between ends 402 and 404. That is, the dimensions and configuration of the contoured distal surface 218 which contacts the epidermis can be adjusted with respect to the arc of the curved (convex) clinical surface to which treatment is to be applied. Of course, this also allows the distance between adjacent cooling units 225a and 228a to be adjusted. However, as described below, in some embodiments the expansion joint 410 is tapered so that the cooling units 225a and 228a maintain proximity at their proximal end in the vicinity of the applicator support 130 while still allowing the distance between cooling units 225a and 228a to be adjusted by the practitioner. As a result there will not be a relatively large intervening segment of adipose tissue that does not receive treatment, which clinically reduces fat cell number and fat roll size in that region.

The expansion joint 410 may be formed from an expandable material that connects one portion of the vacuum cup 210 to its adjacent portion. FIG. 7 shows expansion joint 410 coupling adjacent portions 215 and 217 of the vacuum cup 210. In one embodiment the expansion joint 410 may be integrally formed with the vacuum cup 210 and it may or may not be formed from the same material as the vacuum cup 210. If the expansion joint 410 is formed from the same material as the vacuum cup 210, the expansion joint 410 may be provided with a corrugated or bellows-like configuration (as indicated in FIG. 7) in order to allow it to expand and contract. If the expansion joint 410 is formed from a different material from that of the vacuum cup 210, any suitable material may be selected which is expandable or elastic, yet firm enough to maintain its adherence to the epidermis of the patient so that the vacuum cup 210 does not collapse when a vacuum is applied to its interior cavity 216. In those embodiments in which the expansion joint 410 is not integrally formed with the vacuum cup 210, any suitable means may be used to connect them, including adhesive, fasteners and the like.

As further shown in FIG. 7, in some embodiments the expansion joint 410 begins at the mouth of the vacuum cup 210 and is tapered inward as it extends from the distal end of the vacuum cup 210 toward the proximal end. The expansion joint 410 may or may not fully extend to the proximal end of the vacuum cup 210. Of course, a similar expansion joint (not shown) may be located on the opposing side of the vacuum cup 210, which is not visible in FIG. 7.

While the cooling units 225a and 228a shown in FIG. 7 are square in shape, more generally the cooling units 225a and 228a may be provided with various shapes and sizes and are not limited to the shape and size shown in FIG. 7. For example, the cooling units 225a and 228a may or may not have a curved contour on their distal surfaces such as described above in connection with FIGS. 1-5. Additionally, the cooling units 225a and 228b may or may not have the same size and shape with respect to one another. Moreover, although the expansion joint 410 in FIG. 7 is located between cooling units, in some embodiments one or more expansion joints may be located on either side of the cooling units 225a and 228b, near the end 402 of the vacuum cup 410 and/or near the end 404 of the vacuum cup.

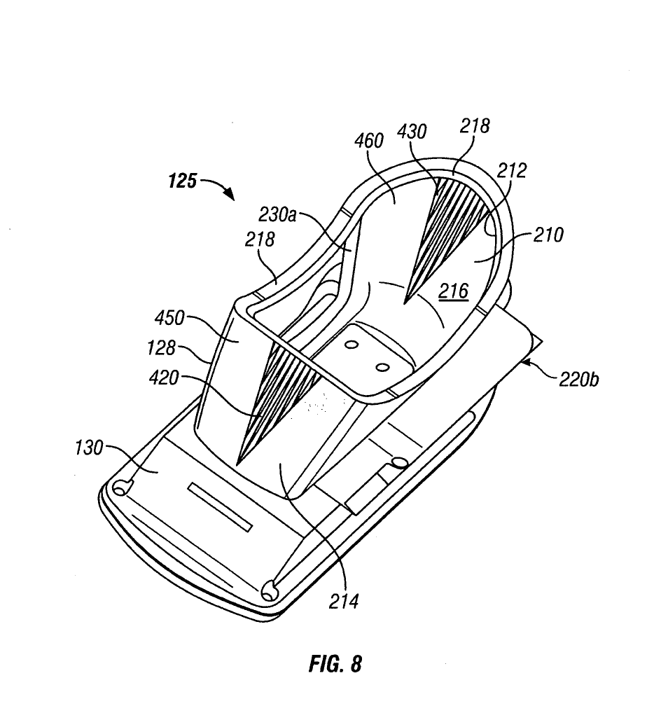

FIG. 8 shows another embodiment of the treatment device 125 having an expandable applicator. In this embodiment outer expansion joints 420 and 430 are located on the side surfaces 450 and 460, respectively, which interconnect the sidewalls of the vacuum cup 210 in which the cooling units are located. The outer expansion joints 420 and 430 can be used by the practitioner to adjust the distance or gap between the cooling units 220a and 220b. In like manner with the expansion joint 410 shown in FIG. 7, outer expansion joints 420 and 430 may be formed from a variety of different expandable or elastic materials and they may be integrally formed with the vacuum cup or, alternatively, attached to adjacent portions of the vacuum cup 210 using any suitable technique and material, such as those discussed above.

In operation, an embodiment according to the present disclosure may include preparing a target area for treatment by applying a sleeve or liner for preventing direct contact between the applicator and a patient's skin, thereby reducing the likelihood of cross-contamination between patients and minimizing cleaning requirements for the applicator. A thermal coupling fluid such as a cryoprotectant gel may be included with the sleeve or liner. Next, the treatment device is applied over the sleeve or liner and treatment may be initiated using the control panel described above. As part of the treatment process, a vacuum may be applied to pull skin and underlying adipose tissue in the target area away from the body.

More specifically, upon receiving input to start a treatment protocol, the processor can cause the treatment device to cycle through one or more segments of a prescribed treatment plan. In so doing, the treatment device applies power to one or more cooling segments, such as TEEs, to begin a cooling cycle and, for example, activate features or modes such as vibration, massage, vacuum, etc. Using temperature or other sensors proximate to the treatment device the processor determines whether a temperature is sufficiently close to the target temperature has been reached. If the target temperature has not been reached, power can be increased or decreased to change the heat flux, as needed, to maintain the target temperature. When the prescribed segment duration expires, the processing unit may apply the temperature and duration indicated in the next treatment profile segment. Additional segments of the plan, if any, are executed by the processor until the treatment protocol is complete.

While the present description provides multiple embodiments and configurations, it should be noted that the present invention is not limited to these embodiments and configurations. Instead, other embodiments and configurations may be provided, as an example, by combining elements of different embodiments. For instance, another embodiment of the treatment device combines the embodiments of FIGS. 7 and 8 to provide an expandable applicator having four expandable joints, each disposed on a different surface of the vacuum cup. Such an embodiment allows the gap between the cooling plates and/or the curve or arc of the treatment zone on a curved clinical surface to be adjusted.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. An applicator for treating lipid-rich cells disposed under a cutaneous layer, comprising:
a vacuum cup defining an interior cavity and including a first concave contour that defines a mouth of the interior cavity;
at least a first cutout extending through a first sidewall of the vacuum cup; and
at least a first cooling unit disposed in the first cutout and having a second concave contour, the cooling unit being configured for heat transfer with respect to the lipid-rich cells when the first and second contours engage the cutaneous layer.

2. The applicator of claim 1 wherein the first and second concave contours have a common curvature.

3. The applicator of claim 2 wherein the cooling unit includes a thermally conductive surface being exposed to the interior cavity of the vacuum cup, said thermally conductive having an edge with a third concave contour matching the second concave contour.

4. The applicator of claim 1 further comprising at least a first expansion joint disposed in the vacuum cup for adjusting at least one dimension of the interior cavity.

5. The applicator of claim 1 further comprising:
a second cutout extending through the sidewall of the vacuum cup; and
a second cooling unit disposed in the second cutout, wherein the first expansion joint is disposed in the vacuum cup between the first and second cooling units.

6. The applicator of claim 4 further comprising:
a second cutout extending through a second sidewall of the vacuum cup; and
a second cooling unit disposed in the second cutout, wherein the first expansion joint is configured to adjust a gap across the mouth of the interior cavity between the first and second cooling units.

7. The applicator of claim 5 wherein the second cooling unit has a third concave contour, wherein the first, second and third concave contours have a common curvature.

8. A treatment device for treating lipid-rich cells disposed under a cutaneous layer, comprising:
a flexible member having an inner and outer surface, the inner surface defining an interior cavity, the flexible member including a first distal surface coupling the inner surface to the outer surface, the first distal surface being configured to engage with the cutaneous layer, the first distal surface having a concave curvature;
at least one cooling unit being coupled to the flexible member, the cooling unit having a thermally conductive member configured to contact the cutaneous layer when the cutaneous layer is drawn into the interior cavity when a vacuum is established therein, the cooling unit having an outer housing with a second distal surface that is also configured to engage with the cutaneous layer when the first distal surface of the flexible member engages with the cutaneous layer, the second distal surface of the outer housing having a concave curvature; and
a support member being coupled to a proximal end of the flexible member.

9. The treatment device of claim 8 wherein the flexible member has a sidewall with a cutout located therein extending through the inner and outer surfaces, the cooling unit being located in the sidewall.

10. The treatment device of claim 9 wherein the thermally conductive member has a first distal edge and the cutout has a second distal edge in contact with the first distal edge of the thermally conductive member, the first distal edge of the thermally conductive member and the second distal edge of the cutout having a common concave curvature.

11. The treatment device of claim 10 wherein the distal surface of the flexible member, the first distal edge of the thermally conductive member and the second distal edge of the cutout all have a common concave curvature.

12. The treatment device of claim 8 further comprising a user control panel located in the support member.

13. The treatment device of claim 8 wherein the cooling unit comprises: at least one thermoelectric cooling unit having a cold side in thermal contact with the thermal conductor and a hot side positioned opposite the cold side; and a heat exchanger in thermal contact with the hot side of the thermoelectric cooling unit.

14. The treatment device of claim 8 further comprising a frame located in the cutout coupling the flexible member to the cooling unit.

15. An applicator for treating lipid-rich cells disposed under a cutaneous layer, comprising:
a vacuum cup having first and second opposing sidewalls defining an interior cavity and including a first distal surface that defines a mouth of the interior cavity;
at least a first cutout extending through the first sidewall of the vacuum cup;
at least a first cooling unit disposed in the first cutout, the cooling unit being configured for heat transfer with respect to the lipid-rich cells when the first distal surface engages the cutaneous layer;
at least a first expansion joint disposed in at least one of the sidewalls of the vacuum cup for adjusting at least one dimension of the interior cavity.

16. The applicator of claim 15 further comprising:
a second cutout extending through the first sidewall of the vacuum cup; and
a second cooling unit disposed in the second cutout, wherein the first expansion joint is disposed in the first sidewall of the vacuum cup between the first and second cooling units.

17. The applicator of claim 15 further comprising:
a second expansion joint located in a third sidewall of the vacuum cup which interconnects the first and second sidewalls, the second expansion joint being configured to adjust a gap across the mouth of the interior cavity between the first and second cooling units.

18. The applicator of claim 16 further comprising:
a second expansion joint disposed in a third sidewall of the vacuum cup which interconnects the first and second sidewalls, the second expansion joint being configured to adjust a gap across the mouth of the interior cavity between the first and second cooling units.

19. The applicator of claim 15 wherein the first cooling unit has surface with a first concave contour and the first distal surface of the vacuum cup has a second concave contour such that the cooling unit is configured for heat transfer with respect to the lipid-rich cells when the first distal surface and the concave surface of the first cooling unit engages the cutaneous layer

20. The applicator of claim 15 wherein the first expansion joint is integrally formed with the vacuum cup.
